(19) United States
(12) Patent Application Publication
(10) Pub. No.: US 2026/0024243 A1
(43) Pub. Date: Jan. 22, 2026

(54) USER INTERFACE FOR STRUCTURES DETECTED IN SURGICAL PROCEDURES

(71) Applicant: DIGITAL SURGERY LIMITED, London (GB)

(72) Inventors: Patrick deKlee, London (GB); Anthony J. Inwood, London (GB)

(21) Appl. No.: 18/993,260

(22) PCT Filed: Jul. 7, 2023
(86) PCT No.: PCT/EP2023/068919

§ 371 Date: Jan. 10, 2025

(30) Foreign Application Priority Data
Jul. 11, 2022 (GR) 20220100548

(51) Int. Cl. G06T 11/00; A61B 90/00; G06V 10/82; G06V 20/40; G06V 20/50; G06V 20/70

(52) U.S. Cl. CPC G06T 11/001; A61B 90/37; G06V 10/82; G06V 20/40; G06V 20/50; G06V 20/70; A61B 2090/373; G06T 2200/24; G06T 2210/41; G06V 2201/031

(57) ABSTRACT

Technical solutions are provided to facilitate computer assistance during a surgery to prevent complications by detecting. identifying. and indicating the identification of certain structures in a field of view of a video of the surgery. According to some aspects. a computer vision system is trained to detect several structures in the video of the surgery, and further to distinguish between the structures. Further, a user interface element is displayed that indicates the identification of the structure by updating a visual attribute of the user interface clement to match a visual attribute used to represent the indicated structure itself.

### BACKGROUND

The present disclosure relates in general to computing technology and relates more particularly to computing technology for automatic detecting structures in surgical procedures using machine learning and providing user feedback based on the automatic detection.

Computer-assisted systems can be useful to augment a person's physical sensing, perception, and reaction capabilities. For example, such systems can effectively provide the information corresponding to an expanded field of vision, both temporal and spatial, that enables a person to adjust current and future actions and decisions based on the part of an environment not included in his or her physical field of view. Additionally, the systems can bring attention to occluded parts of the view, for example, due to structures, blood, etc. However, providing such information relies upon an ability to process part of this extended field in a useful manner. Highly variable, dynamic, and/or unpredictable environments present challenges in defining rules that indicate how representations of the environments are to be processed to output data to productively assist the person in action performance.

### SUMMARY

According to one or more aspects, a computer-implemented user interface includes a plurality of user interface elements, each user interface element respectively corresponding to a structure from a list of structures anticipated in a surgical video, each user interface element having a visual attribute, wherein the visual attribute of a first user interface element is set to a first state in response to a first structure being detected in the surgical video in a field of view, and the visual attribute of the first user interface element is set to a second state in response to the first structure not being detected in the field of view.

According to an aspect, the visual attribute of the first user interface element is based on the first structure being marked with an overlay using said visual attribute.

According to an aspect, the first structure is detected in the field of view using machine learning.

According to an aspect, the plurality of user interface elements is grouped in a single menu user interface. According to an aspect, the menu user interface is a toolbar displayed as a graphical overlay on the surgical video. According to an aspect, the surgical video is a live video stream. According to an aspect, a location of rendering the toolbar is fixed or user-configurable.

According to an aspect, the list of structures comprises a predetermined list of structures based on a type of surgery in the surgical video.

According to an aspect, the list of structures comprises a dynamic list of structures.

According to an aspect, the visual attribute is one of a color, a pattern, a shape, an image, and an animation.

According to an aspect, the user interface element comprises a label.

According to one or more aspects, a computer-implemented method includes identifying, by one or more processors, a structure in a video of a surgical procedure using machine learning. The method further includes, in response to at least a portion of the structure being visible in a field of view, representing, by the one or more processors, a user interface element corresponding to the structure using a first visual attribute. The method further includes, in response to the structure not being visible in the field of view, representing, by the one or more processors, the user interface element corresponding to the structure using a second visual attribute.

According to an aspect, the structure is one of an anatomical structure and a surgical instrument.

According to an aspect, the anatomical structure is one of an organ, artery, duct, surgical artifact, and anatomical landmark.

According to an aspect, the surgical instrument is one of clamps, staplers, knives, scalpels, sealers, dividers, dissectors, tissue fusion instruments, monopolars, Marylands, and fenestrated.

According to an aspect, the structure is one from a predetermined list of structures.

According to an aspect, the symbol comprises a geometric shape that is displayed at a predetermined position during a display of the video of the surgical procedure.

According to an aspect, the first visual attribute is used to represent the symbol in response to the structure being the field of view based on the first visual attribute being used to highlight the structure.

According to an aspect, the video is a live video stream of the surgical procedure.

According to one or more aspects, a computer program product includes a memory device having computer executable instructions stored thereon, which when executed by one or more processors cause the one or more processors to perform a method for generating a user interface to depict presence of structures in a field of view. The method includes identifying, using a neural network model, a structure in a video of a surgical procedure, the neural network model is trained using surgical training data. The method further includes generating a visualization that comprises a graphical overlay at a location of the structure in the video of the surgical procedure, the graphical overlay uses a first visual attribute. The method further includes identifying a symbol corresponding to the structure from a list of displayed symbols. The method further includes updating the symbol by displaying the symbol using the first visual attribute.

Additional technical features and benefits are realized through the techniques of the present invention. Aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 shows an example snapshot of a laparoscopic cholecystectomy being performed;

FIG. 2 shows a system for detecting structures in surgical data using machine learning according to one or more aspects;

FIG. 3 depicts a flowchart of a method for generating a user interface according to one or more aspects;

FIG. 4 depicts a computer system in accordance with one or more aspects; and

FIG. 5 depicts a surgical procedure system in accordance with one or more aspects.

The diagrams depicted herein are illustrative. There can be many variations to the diagram, or the operations described therein without departing from the spirit of the invention. For instance, the actions can be performed in a differing order, or actions can be added, deleted, or modified. Also, the term “coupled,” and variations thereof describe having a communications path between two elements and do not imply a direct connection between the elements with no intervening elements/connections between them. All of these variations are considered a part of the specification.

### DETAILED DESCRIPTION

Exemplary aspects of technical solutions described herein relate to, among other things, devices, systems, methods, computer-readable media, techniques, and methodologies for using machine learning and computer vision to improve surgical safety and workflow by automatically detecting one or more structures in surgical data. The structures may be deemed to be critical for an actor involved in performing one or more actions during a surgical procedure (e.g., by a surgeon) in some aspects. In one or more aspects, the structures are detected dynamically and in real-time as the surgical data is being captured by technical solutions described herein. A detected structure can be an anatomical structure, a surgical instrument, etc. Further, aspects of the technical solutions described herein address the technical challenge of distinguishing between structures and indicating to the actor an identity of the structure that is identified.

Description of the technical solutions herein is provided using one or more particular surgical procedures, such as laparoscopic cholecystectomy, as examples. However, it should be appreciated that the technical solutions described herein are not limited to only those types of surgical procedures. The technical solutions described herein are applicable to any other type of surgical procedure where identification of structures and clearly depicting to an actor the identified structures is helpful.

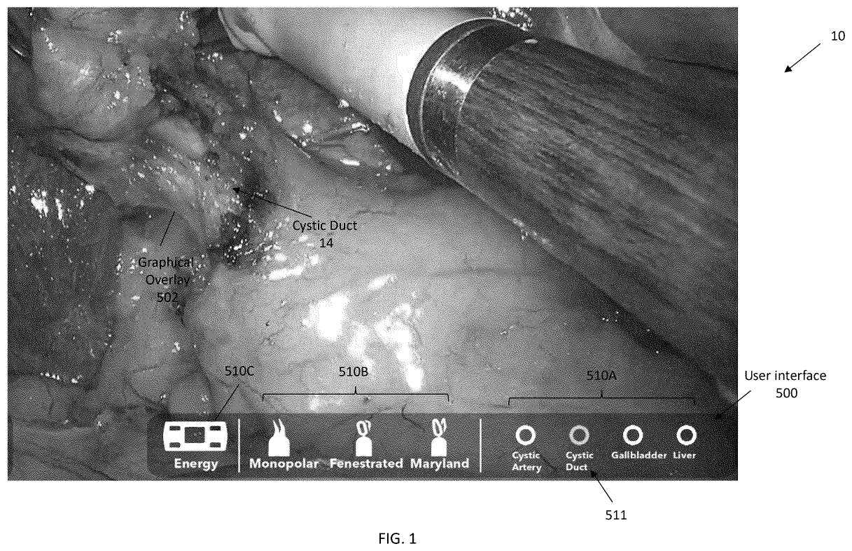

Laparoscopic cholecystectomy is a common surgery in which the gallbladder is removed. This involves exposing one or more anatomical structures, such as the gallbladder, the liver, the cystic duct, the cystic artery, etc. The procedure may include clipping and dividing one or more structures and then extracting the gallbladder. FIG. 1 shows an example snapshot 10 of laparoscopic cholecystectomy, an anatomical structure labeled. In snapshot 10, shown in FIG. 1, the cystic duct 14 is labeled. As can be seen, the anatomical structures can be difficult to distinguish by visual cues without context, such as the direction of viewing, etc. Complications can occur when the structures are misidentified or confused with other structures in the vicinity. For example, the cystic artery can be in the vicinity of the cystic duct 14, particularly as they may be difficult to distinguish without thorough dissection.

In some instances, a computer-assisted surgical (CAS) system is provided that uses one or more machine learning models, trained with surgical data, to augment environmental data directly sensed by an actor involved in performing one or more actions during a surgical procedure (e.g., a surgeon). Such augmentation of perception and action can increase action precision, optimize ergonomics, improve action efficacy, enhance patient safety, and improve the standard of the surgical process.

The surgical data provided to train the machine learning models can include data captured during a surgical procedure, as well as simulated data. The surgical data can include time-varying image data (e.g., a simulated/real video stream from several types of cameras) corresponding to a surgical environment. The surgical data can also include other types of data streams, such as audio, radio frequency identifier (RFID), text, robotic sensors, other signals, etc. The machine learning models are trained to detect and identify, in the surgical data, “structures,” including particular tools, anatomical objects, and actions being performed in the simulated/real surgical stages. In one or more aspects, the machine learning models are trained to define one or more parameters of the models so as to learn how to transform new input data (that the models are not trained on) to identify one or more structures. During the training, the models are input one or more data streams that may be augmented with data indicating the structures in the data streams, such as indicated by metadata and/or image-segmentation data associated with the input data. The data used during training can also include temporal sequences of one or more input data.

In one or more aspects, the simulated data can be generated to include image data (e.g., which can include time-series image data or video data and can be generated in any wavelength of sensitivity) that is associated with variable perspectives, camera poses, lighting (e.g., intensity, hue, etc.) and/or motion of imaged objects (e.g., tools). In some instances, multiple data sets can be generated—each of which corresponds to the same imaged virtual scene but varies with respect to perspective, camera pose, lighting, and/or motion of imaged objects, or varies with respect to the modality used for sensing, e.g., red-green-blue (RGB) images or depth or temperature. In some instances, each of the multiple data sets corresponds to a different imaged virtual scene and further varies with respect to perspective, camera pose, lighting, and/or motion of imaged objects.

The machine learning models can include a fully convolutional network adaptation (FCN) and/or conditional generative adversarial network model configured with one or more hyperparameters to perform image segmentation into classes. For example, the machine learning models (e.g., the fully convolutional network adaptation) can be configured to perform supervised, self-supervised, or semi-supervised semantic segmentation in multiple classes—each of which corresponding to a particular surgical instrument, anatomical body part (e.g., generally or in a particular state), and/or environment. Alternatively, or in addition, the machine learning model (e.g., the conditional generative adversarial network model) can be configured to perform unsupervised domain adaptation to translate simulated images to semantic segmentation. In one or more aspects, the machine learning model uses a neural network architecture of DeepLabV3+ and ResNet101 encoder. It is understood that other types of machine learning models or combinations thereof can be used in one or more aspects.

The trained machine learning model can then be used in real-time to process one or more data streams (e.g., video streams, audio streams, RFID data, etc.). The processing can include detecting and characterizing one or more structures within various instantaneous or block time periods. The structure(s) can then be used to identify the presence, position, and/or use of one or more features. Alternatively, or in addition, the structures can be used to identify a stage within a workflow (e.g., as represented via a surgical data structure), predict a future stage within a workflow, etc.

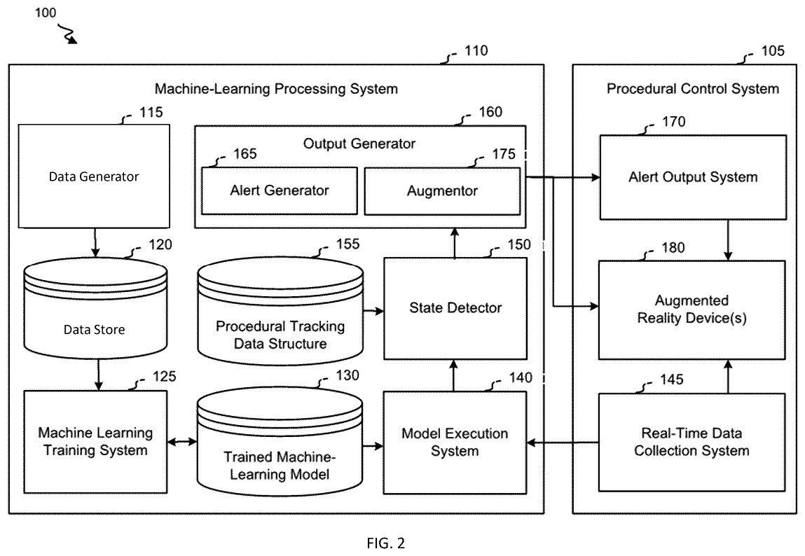

FIG. 2 shows a system 100 for detecting structures in surgical data using machine learning according to one or more aspects. System 100 uses data streams in the surgical data to identify procedural states according to some aspects. System 100 includes a procedural control system 105 that collects image data and coordinates outputs responsive to detected structures and states. The procedural control system 105 can include one or more devices (e.g., one or more user devices and/or servers) located within and/or associated with a surgical operating room and/or control center. System 100 further includes a machine learning processing system 110 that processes the surgical data using a machine learning model to identify a procedural state (also referred to as a phase or a stage), which is used to identify a corresponding output. It will be appreciated that machine learning processing system 110 can include one or more devices (e.g., one or more servers), each of which can be configured to include part or all of one or more of the depicted components of the machine learning processing system 110. In some instances, a part, or all of machine learning processing system 110 is in the cloud and/or remote from an operating room and/or physical location corresponding to a part, or all of procedural control system 105. For example, the machine learning training system 125 can be a separate device (e.g., server) that stores its output as the one or more trained machine learning models 130, which are accessible by the model execution system 140, separate from the machine learning training system 125. In other words, in some aspects, devices that “train” the models are separate from devices that “infer,” i.e., perform real-time processing of surgical data using the trained models 130.

Machine learning processing system 110 includes a data generator 115 configured to generate simulated surgical data, such as a set of virtual images, or record surgical data from ongoing procedures, to train a machine learning model. Data generator 115 can access (read/write) a data store 120 with recorded data, including multiple images and/or multiple videos. The images and/or videos can include images and/or videos collected during one or more procedures (e.g., one or more surgical procedures). For example, the images and/or video may have been collected by a user device worn by a participant (e.g., surgeon, surgical nurse, anesthesiologist, etc.) during the surgery, and/or by a non-wearable imaging device located within an operating room.

Each of the images and/or videos included in the recorded data can be defined as a base image and can be associated with other data that characterizes an associated procedure and/or rendering specifications. For example, the other data can identify a type of procedure, a location of a procedure, one or more people involved in performing the procedure, and/or an outcome of the procedure. Alternatively, or in addition, the other data can indicate a stage of the procedure with which the image or video corresponds, rendering specification with which the image or video corresponds, and/or a type of imaging device that captured the image or video (e.g., and/or, if the device is a wearable device, a role of a particular person wearing the device, etc.). Further, the other data can include image-segmentation data that identifies and/or characterizes one or more objects (e.g., tools, anatomical objects, etc.) that are depicted in the image or video. The characterization can indicate the position, orientation, or pose of the object in the image. For example, the characterization can indicate a set of pixels that correspond to the object and/or a state of the object resulting from a past or current user handling.

Data generator 115 identifies one or more sets of rendering specifications for the set of virtual images. An identification is made as to which rendering specifications are to be specifically fixed and/or varied. Alternatively, or in addition, the rendering specifications that are to be fixed (or varied) are predefined. The identification can be made based on, for example, input from a client device, a distribution of one or more rendering specifications across the base images and/or videos, and/or a distribution of one or more rendering specifications across other image data. For example, if a particular specification is substantially constant across a sizable data set, the data generator 115 defines a fixed corresponding value for the specification. As another example, if rendering-specification values from at least a predetermined amount of data span across a range, the data generator 115 defines the rendering specifications based on the range (e.g., to span the range or to span another range that is mathematically related to the range of distribution of the values).

A set of rendering specifications can be defined to include discrete or continuous (finely quantized) values. A set of rendering specifications can be defined by a distribution, such that specific values are to be selected by sampling from the distribution using random or biased processes.

One or more sets of rendering specifications can be defined independently or in a relational manner. For example, if the data generator 115 identifies five values for a first rendering specification and four values for a second rendering specification, the one or more sets of rendering specifications can be defined to include twenty combinations of the rendering specifications or fewer (e.g., if one of the second rendering specifications is only to be used in combination with an incomplete subset of the first rendering specification values or the converse). In some instances, different rendering specifications can be identified for different procedural phases and/or other metadata parameters (e.g., procedural types, procedural locations, etc.).

Using the rendering specifications and base image data, the data generator 115 generates simulated surgical data (e.g., a set of virtual images), which is stored at the data store 120. For example, a three-dimensional model of an environment and/or one or more objects can be generated using the base image data. Virtual image data can be generated using the model to determine—given a set of particular rendering specifications (e.g., background lighting intensity, perspective, zoom, etc.) and other procedure-associated metadata (e.g., a type of procedure, a procedural state, a type of imaging device, etc.). The generation can include, for example, performing one or more transformations, translations, and/or zoom operations. The generation can further include adjusting the overall intensity of pixel values and/or transforming RGB values to achieve particular color-specific specifications.

A machine learning training system 125 uses the recorded data in the data store 120, which can include the simulated surgical data (e.g., a set of virtual images) and actual surgical data to train one or more machine learning models. The machine learning models can be defined based on a type of model and a set of hyperparameters (e.g., defined based on input from a client device). The machine learning models can be configured based on a set of parameters that can be dynamically defined based on (e.g., continuous, or repeated) training (i.e., learning, parameter tuning). Machine learning training system 125 can use one or more optimization algorithms to define the set of parameters to minimize or maximize one or more loss functions. The set of (learned) parameters can be stored at a trained machine learning model data structure 130, which can also include one or more non-learnable variables (e.g., hyperparameters and/or model definitions).

A model execution system 140 can access the machine learning model data structure 130 and accordingly configure a machine learning model for inference (i.e., detection). The machine learning model can include, for example, a fully convolutional network adaptation, an adversarial network model, or other types of models as indicated in data structure 130. The machine learning model can be configured in accordance with one or more hyperparameters and the set of learned parameters.

The machine learning model, during execution, receives, as input, surgical data to be processed and generates an inference according to the training. For example, the surgical data can include data streams (e.g., an array of intensity, depth, and/or RGB values) for a single image or for each of a set of frames representing a temporal window of fixed or variable length in a video. The surgical data that is input can be received from a real-time data collection system 145, which can include one or more devices located within an operating room and/or streaming live imaging data collected during the performance of a procedure. The surgical data can include additional data streams such as audio data, RFID data, textual data, measurements from one or more instruments/sensors, etc., that can represent stimuli/procedural states from the operating room. The different inputs from different devices/sensors are synchronized before inputting into the model.

The machine learning model analyzes the surgical data and, in one or more aspects, detects and/or characterizes structures included in the visual data from the surgical data. The visual data can include image and/or video data in the surgical data. The detection and/or characterization of the structures can include segmenting the visual data or detecting the localization of the structures with a probabilistic heatmap. In some instances, the machine learning model includes or is associated with a preprocessing or augmentation (e.g., intensity normalization, resizing, cropping, etc.) that is performed prior to segmenting the visual data. An output of the machine learning model can include image-segmentation or probabilistic heatmap data that indicates which (if any) of a defined set of structures are detected within the visual data, a location and/or position, and/or pose of the structure(s) within the image data, and/or state of the structure(s). The location can be a set of coordinates in the image data. For example, the coordinates can provide a bounding box. Alternatively, the coordinates provide boundaries that surround the structure(s) being detected.

A state detector 150 can use the output from the execution of the machine learning model to identify a state within a surgical procedure (“procedure”). A procedural tracking data structure can identify a set of potential states that can correspond to part of a performance of a specific type of procedure. Different procedural data structures (e.g., different machine learning-model parameters and/or hyperparameters) may be associated with different types of procedures. The data structure can include a set of nodes, with each node corresponding to a potential state. The data structure can include directional connections between nodes that indicate (via the direction) an expected order during which the states will be encountered throughout an iteration of the procedure. The data structure may include one or more branching nodes that feed to multiple next nodes and/or can include one or more points of divergence and/or convergence between the nodes. In some instances, a procedural state indicates a surgical action that is being performed or has been performed and/or indicates a combination of actions that have been performed. A “surgical action” can include an operation such as an incision, a compression, a stapling, a clipping, a suturing, a cauterization, a sealing, or any other such actions performed to complete a step/phase in the surgical procedure. In some instances, a procedural state relates to a biological state of a patient undergoing a surgical procedure. For example, the biological state can indicate a complication (e.g., blood clots, clogged arteries/veins, etc.) or precondition (e.g., lesions, polyps, etc.).

Each node within the data structure can identify one or more characteristics of the state. The characteristics can include visual characteristics. In some instances, the node identifies one or more tools that are typically in use or availed for use (e.g., on a tool try) during the state, one or more roles of people who are typically performing a surgical task, a typical type of movement (e.g., of a hand or tool), etc. Thus, state detector 150 can use the segmented data generated by model execution system 140 that indicates the presence and/or characteristics of particular objects within a field of view to identify an estimated node to which the real image data corresponds. Identification of the node (and/or state) can further be based upon previously detected states for a given procedural iteration and/or other detected input (e.g., verbal audio data that includes person-to-person requests or comments, explicit identifications of a current or past state, information requests, etc.).

An output generator 160 can use the state to generate an output. Output generator 160 can include an alert generator 165 that generates and/or retrieves information associated with the state and/or potential next events. For example, the information can include details as to warnings and/or advice corresponding to current or anticipated procedural actions. The information can further include one or more events for which to monitor. The information can identify the next recommended action.

The user feedback can be transmitted to an alert output system 170, which can cause the user feedback to be output via a user device and/or other devices that is (for example) located within the operating room or control center. The user feedback can include a visual, audio, tactile, or haptic output that is indicative of the information. The user feedback can facilitate alerting an operator, for example, a surgeon or any other user of the system.

Output generator 160 can also include an augmentor 175 that generates or retrieves one or more graphics and/or text to be visually presented on (e.g., overlaid on) or near (e.g., presented underneath or adjacent to or on a separate screen) in real-time capture of a procedure. Augmentor 175 can further identify where the graphics and/or text are to be presented (e.g., within a specified size of a display). In some instances, a defined part of a field of view is designated as being a display portion to include augmented data. In some instances, the position of the graphics and/or text is defined so as not to obscure the view of an important part of an environment for the surgery and/or to overlay particular graphics (e.g., of a tool) with the corresponding real-world representation.

Augmentor 175 can send the graphics and/or text and/or any positioning information to an augmented reality device 180, which can integrate the graphics and/or text with a user's environment in real-time as an augmented visualization. Augmented reality device 180 can include a pair of goggles that can be worn by a person participating in part of the procedure. It will be appreciated that, in some instances, the augmented display can be presented on a non-wearable user device, such as a computer or tablet. The augmented reality device 180 can present the graphics and/or text at a position as identified by augmentor 175 and/or at a predefined position. Thus, a user can maintain a real-time view of procedural operations and further view pertinent state-related information.

Presently, existing solutions provide official guidance that requires surgeons to establish a “critical view of safety” (CVS) before clipping and division. In CVS, both structures can clearly and separately be identified and traced as they enter the gallbladder. Some existing techniques create a bounding box detection system based on anatomical landmarks that include the common bile duct and the cystic duct 14 but not the cystic artery. Existing solutions provide different techniques, including machine learning techniques to detect and identify one or more of the anatomical structures in an input image. However, a technical challenge exists in conveying, to an actor, information about the structure that is identified.

In existing solutions, the identified structures are marked, for example, using graphical overlays, such as the graphical overlay 502 shown in FIG. 1. Here, “marking” an anatomical structure, surgical instrument, or other features in the surgical data includes visually highlighting that feature for the surgeon or any other user by using the graphical overlay 502. The graphical overlay 502 can include a heatmap, a contour, a bounding box, a mask, a highlight, or any other such visualization that is overlaid on image 10 that is being displayed to the user. Further, in one or more aspects, the specific anatomical structures that are identified are marked using predetermined values that are assigned to respective anatomical structures. For example, as shown in FIG. 1, the cystic duct 14 is marked using a first color value (e.g., green), and the cystic duct may be marked using a second color value (e.g., purple), and so on. It can be appreciated that visual attributes other than color or a combination thereof can also be assigned to specific structures. The assignment of the visual attributes to respective structures can be user-configurable. The examples herein depict using masks and heatmaps as the graphical overlays 502. However, different techniques can be used in other aspects. Various visual attributes of the graphical overlay 502, such as colors, transparency, visual pattern, line thickness, etc., can be adjusted.

To identify the type of the structure that is identified, existing solutions display an annotation in addition to the graphic overlay 502. The annotation can be a text label that identifies the name of the structure, for example, anatomical structure(s) or other objects. However, a technical challenge exists where such annotations may not be visible as the structure changes in position in the view. Further, such annotations can cover portions of the surgical data that the user may desire to see or are critical for the user to see. Additionally, when multiple structures are identified, the respective annotations for the structures can occupy an undesirable amount of the view. Additionally, the multiple annotations may overlap, making them visually illegible and also aesthetically unappealing.

Technical solutions described herein provide a user interface that addresses such technical challenges and provides several technical improvements to surgery systems. Technical solutions described herein provide a dynamic index of structures that includes a list of symbols respectively corresponding to a list of structures. A symbol in the index is highlighted using a visual attribute (e.g., color) that matches the visual attribute of the graphical overlay 502 used to mark the structure corresponding to the symbol. The symbol is highlighted only when the corresponding structure is in the field of view, is identified, and is marked (e.g., using graphical overlay 502). When the structure is not in view, the symbol is not highlighted. While aspects of the technical solutions herein are described as detecting a “structure” being within (or outside) the field of view, it should be noted that the entirety of the structure may not be detected/identified in some aspects. In some aspects, only a portion of the structure being within (or outside) the field of view can cause the symbol to be highlighted (or not). The portion that is identified is a predetermined portion in one or more aspects. Accordingly, the surgical field of view stays free of annotations and labels, and only the structures such as anatomical structures, surgical instruments, etc., stay visible to the actor in the surgical field of view. The index assists in identifying the structure.

As shown in FIG. 1, aspects described herein facilitate generating a computer-implemented user interface 500. The user interface 500 can be a toolbar, a menu bar, or any other such collection of user interface elements (510), in some aspects. In other aspects, the user interface 500 can be a single user interface element (510), and a plurality of such user interfaces 500 are used. FIG. 1 depicts several types of user interface elements (UIE) 510—a first UIE type 510A, a second UIE type 510B, and a third UIE type 510C—collectively referred to as UIEs 510.

In some aspects, each user interface element 510 includes a symbol 515 that corresponds to a specific structure that can be identified in the video of the surgical procedure. In some aspects, the UIE 510 can also include an annotation 516 (textual name/description of the corresponding structure). Other components can be included in the UIE 510 in some aspects which are not shown herein

In some aspects, UIE 510 of a specific type, i.e., corresponding to a specific type of structure, are grouped together. For example, the first UIE type 510A can represent anatomical structures detected and identified in the video; 510B can represent surgical instruments detected and identified in the video; and 510C can represent a state of an energy platform (e.g., Valleylab™ FT10 Energy Platform). It is understood that the grouping and sequence of the UIE 510 can be different from what is shown in FIG. 1 in other aspects.

In some aspects, a visual attribute of a first UIE 510, say 511, is set to a first state/value in response to a first structure, say cystic duct 14, being detected in the surgical video in a field of view. The visual attribute of the first UIE 511 is set to a second state/value in response to the first structure not being detected in the field of view. Accordingly, the UIE 511 is highlighted when the corresponding structure is in the field of view and is not highlighted when the corresponding structure is not in the field of view. The highlighting (or not) can be achieved by changing/updating a visual attribute of the symbol 515 and/or the annotation 516.

In some aspects, the visual attribute used to highlight the UIE 511 is based on the visual attribute used to depict the graphical overlay 502 representing the first structure (cystic duct 14); i.e., the graphical overlay 502 and the UIE 511 are represented using the same visual attribute(s). Examples of the visual attribute that are updated to depict the identification of a structure in the field of view are color (e.g., foreground, background, border, etc.), pattern, shape, size, icon, image, and other such attributes and a combination thereof. In one or more aspects, the UIE 511 includes an attribute that facilitates animating the UIE 511. For example, the UIE 511 can depict a flashing color, a glow, or any other visual cue to draw a user's attention. Additionally, or alternatively, in other aspects, the along with the visual attribute of the UIE 511, another sensory cue may be provided to the user. For example, an audible cue such as, a notification tone, may be emitted. In some aspects, haptic feedback may be provided. The haptic feedback may be provided via the surgical instrument that is being used.

The user interface 500 is depicted at a predetermined fixed location on display in some aspects. The position may be based on a predetermined configuration of the display that dictates the locations of one or more components, such as the user interface 500, the video playback area, and other components (e.g., menu, etc., not shown). In some aspects, the location of the user interface 500 can be updated by the user dynamically. For example, the user may move the user interface 500.

In some aspects, the user interface 500 is populated with the UIEs 510 based on the type of surgery being performed. Based on the type of surgery, the particular structures that can be predictably detected in the surgical video of the surgery by using machine learning are listed to be identified in the user interface 500. Alternatively, or in addition, the list of structures to be identified and indicated can be a list of critical structures associated with the type of surgery. Alternatively, or in addition, the user can provide a list of structures that are to be identified by the user interface 500. Based on the list of structures for which identification is to be represented, the user interface 500 is populated with corresponding UIEs 510.

The structures that are to be identified and the identification represented by the user interface 500 can include anatomical structures, surgical instruments, etc. The identification of the structures is performed automatically by using one or more machine learning models that may be known or to be developed.

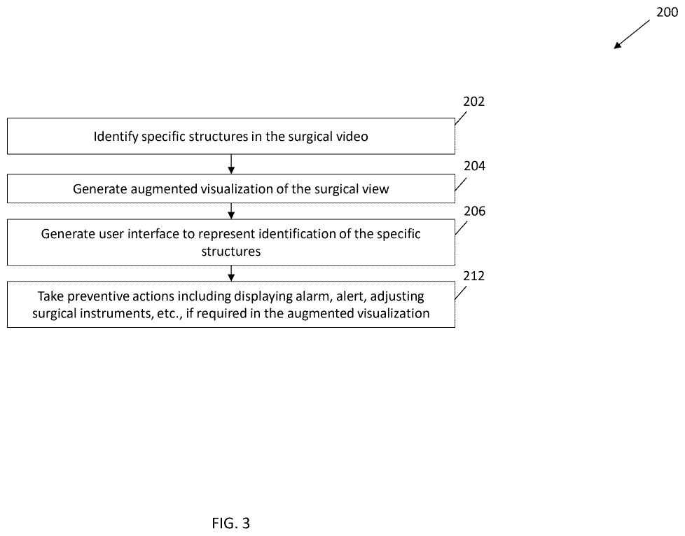

FIG. 3 depicts a flowchart of a method 200 for generating and displaying a user interface that indicates an identification of one or more structures in a surgical video, which are in a field of view according to one or more aspects. Method 200 can be executed by system 100 as a computer-implemented method.

Method 200 includes training and using (inference phase) machine learning model(s) 130 to detect structures in a surgical video, at block 202. The surgical video can be a live video stream in some examples. In other aspects, the surgical video can be a playback of a recorded video. Artificial deep neural networks (DNN), or other types of machine learning models, can be used to achieve automatic, accurate structure detection and identification in surgical procedures, such as cataract surgery, laparoscopic cholecystectomy, endoscopic endonasal transsphenoidal approach (eTSA) to resection of pituitary adenomas, or any other surgical procedure.

The machine learning model(s) 130 includes a feature encoder to detect features from the surgical data for the procedure. The feature encoder can be based on one or more artificial neural networks, such as a convolutional neural network (CNN), a recurrent neural network (RNN), a feature pyramid network (FPN), a transformer network, or any other type of neural network or a combination thereof. The feature encoder can use a known technique, supervised, self-supervised, or unsupervised (e.g., autoencoder), to learn efficient data “codings” in the surgical data. The “coding” maps input data to a feature space, which can be used by feature decoders to perform semantic analysis of the surgical data. In one or more aspects, the machine learning model includes task-specific decoders that detect instruments being used at an instance in the surgical data based on the detected features.

The structures that are detected can include anatomical structures, surgical instruments, and other such features in the surgical features. Anatomical structures that are detected can include organs, arteries, ducts, implants, surgical artifacts (e.g., staples, stitches, etc.), etc. Further yet, based on the type of surgical procedure being performed, one or more of the detected anatomical structures can be identified as critical structures for the success of the procedure. The surgical instruments that are detected can include clamps, staplers, knives, scalpels, sealers, dividers, dissectors, tissue fusion instruments, monopolars, Marylands, fenestrated, etc.

The machine learning model 130 can detect and identify whether a particular structure is (or not) within a field of view. The machine learning model 130 can further indicate a location (in the input image(s)) where the structure is detected and identified.

Method 200 of FIG. 3 further includes generating an augmented visualization of the surgical video using the information obtained from the processing at block 204. The augmented visualization can include, for example, displaying graphical overlays 502 over one or more identified structures in the surgical video. The graphical overlays 502 can represent segmentation masks or probability maps, etc. (FIG. 1 depicts example augmented visualizations of surgical views generated according to one or more aspects. It is understood that those shown are examples and that various other augmented visualizations can be generated in other aspects.

Further, according to a phase of the surgical procedure, the critical anatomical structures that are identified also change. For example, in eye surgery, the iris and a specific portion of the iris that is to be operated on may be the only structures that are to be identified structures using a graphical overlay 502. The sclera, which may also be seen (i.e., in the field of view), may not be marked, for example, because it may not be deemed as a “critical structure” for the surgical procedure or surgical phase being performed.

A user can configure which detections from the machine learning system 100 are to be displayed by the augmentor 175. For example, the user can configure to display overlays 502 on a partial set of the identifications, with the other identifications not being marked in the augmented reality device 180.

“Critical anatomical structures” can be specific to the type of surgical procedure being performed and identified automatically. Additionally, the surgeon or any other user can configure the system 100 to identify particular anatomical structures as critical for a particular patient. The selected anatomical structures are critical to the success of the surgical procedure, such as anatomical landmarks (e.g., Calot triangle, Angle of His, cystic artery 12, cystic duct 14, etc.) that need to be identified during the procedure or those resulting from a previous surgical task or procedure (e.g., stapled, or sutured tissue, clips, etc.).

In some aspects, the surgical instruments in the surgical video may also be marked using graphical overlays 502. The surgical instruments are identified by the machine learning models, as described herein.

In one or more aspects, a user can adjust the attributes of the graphic overlays 502. For example, the user can select a type of highlighting, a color, a line thickness, transparency, a shading pattern, a label, an outline, or any other such attributes to be used to generate and display the graphical overlay on the surgical video. In some aspects, the color and/or transparency of the graphical overlay 502 is modulated based on a confidence score associated with the identification of the underlying anatomical structure or surgical instrument by the machine learning model(s).

In some aspects, in response to the structure visible in the field of view being identified as a structure for which identification is to be indicated, the UIE 510 corresponding to the structure is highlighted at block 206. The highlighting is performed using the visual attribute being used to depict the graphical overlay 502 of the structure. In response to the structure no longer being in the field of view (or not being in the field of view), the corresponding UIE 510 is updated to remove the highlighting (or to be displayed without highlighting). A UIE 510 is depicted/displayed without highlighting when it is displayed using default settings/values for one or more visual attributes of the UIE 510. In some aspects, the settings of the visual attributes used to depict the UIE 510 in the highlighted state can be referred to as a first state/first visual attribute setting, etc. the settings of the visual attributes used to depict the UIE 510 in the non-highlighted state can be referred to as a second state/second visual attribute setting, etc.

In some aspects, two or more of the UIEs 510 can be highlighted at the same time. For example, if two or more structures are in the field of view, each respective corresponding UIE 510 is highlighted. For example, in FIG. 6 another exemplary user interface 500 is depicted. The UIEs 510 shown use different visual elements than those in FIG. 1. It is understood that the depicted examples herein are illustrative, and that in one or more aspects, the UIEs 510 can use different shapes, forms, attributes than those depicted herein. For example, in FIG. 6, view 610 includes the user interface 500 in which each UIE 511 includes an oval symbol 515 and annotation 516. The fill-color (or pattern, etc.) of the symbol 515 is used to as the visual attribute to depict graphical overlay 502 of corresponding structure (e.g., prostate, seminal vesicles, bladder, etc.). Another attribute can be used in other aspects.

View 620 depicts another example of the user interface 500. In this example, the UIE 511 includes the annotation 516, but does not include the symbol 515 (as in other examples). Instead, the visual attributes of the UIE 511 itself are used to represent the graphical overlay 502 corresponding to the structure associated with the UIE 511. For example, here, the fill-color (or fill-patter, etc.) of the UIE 511 is used to represent the graphical overlay 502. (For example, the graphical overlay 502 is of the same color as used for the fill-color). It is understood that the illustrations herein are not limiting, and that other attributes of the UIE 511 can be used to indicate the relationship with the corresponding graphical overlay(s) 502.

Aspects of the technical solutions described herein improve surgical procedures by improving the safety of the procedures. Further, the technical solutions described herein facilitate improvements to computing technology, particularly computing techniques used during a surgical procedure. Aspects of the technical solutions described herein facilitate one or more machine learning models, such as computer vision models, to process images obtained from a live video feed of the surgical procedure in real-time using spatio-temporal information. The machine learning models use techniques such as neural networks to use information from the live video feed and (if available) robotic sensor platform to detect and distinguish one or more features, such as anatomical structures, or surgical instruments, in an input window of the live video feed, and further depict the predictions/identifications to a user in a non-obtrusive, informative, intuitive, configurable, and aesthetically pleasing way. It should be noted that an output of a machine learning model can be referred to as “prediction” unless specified otherwise.

The predictions are used to generate and display graphical overlays to the surgeon and/or other users in an augmented visualization of the surgical view. The graphical overlays can mark critical anatomical structures, surgical instruments, surgical staples, scar tissue, results of previous surgical actions, etc. The graphical overlays can further show a relationship between the surgical instrument(s) and one or more anatomical structures in the surgical view and thus, guide the surgeon and other users during the surgery. The graphical overlays are adjusted according to the user's preferences and/or according to the confidence scores of the predictions.

By using machine learning models, and computing technology to predict and mark various features in the surgical view, in real-time, aspects of the technical solutions facilitate the surgeons to replace visualizations based on external contrast agents (e.g., Indocyanine green (ICG), Ethiodol, etc.) that have to be injected into the patient. Such contrast agents may not always be available to use because of the patient's preconditions or other factors. Accordingly, aspects of the technical solutions described herein provide a practical application in surgical procedures. In some aspects, the contrast agents can be used in addition to the technical solutions described herein. The operator, for example, the surgeon, can switch on/off either (or both) visualizations, the contrast agent based, or the graphical overlays 502.

Further yet, aspects of the technical solutions described herein address the technical challenges of predicting complex features in a live video feed of a surgical view in real-time. The technical challenges are addressed by using real-time analysis and augmented visualization of the surgical view.

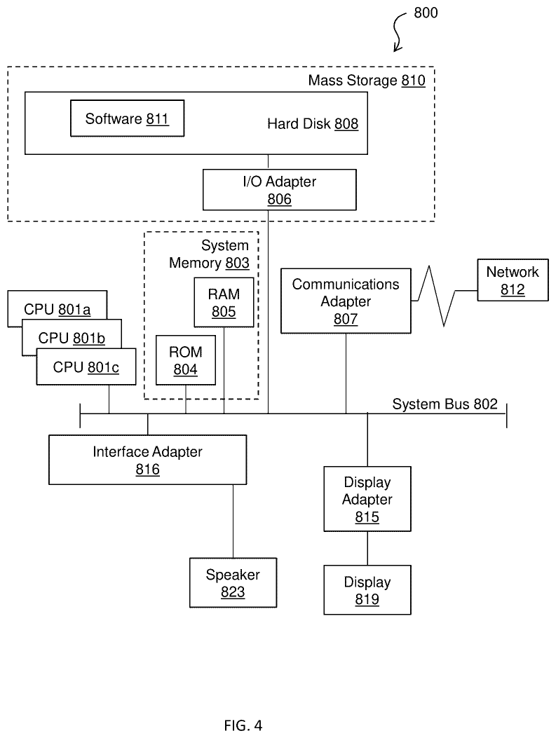

Turning now to FIG. 4, a computer system 800 is generally shown in accordance with an aspect. The computer system 800 can be an electronic computer framework comprising and/or employing any number and combination of computing devices and networks utilizing various communication technologies, as described herein. The computer system 800 can be easily scalable, extensible, and modular, with the ability to change to different services or reconfigure some features independently of others. The computer system 800 may be, for example, a server, desktop computer, laptop computer, tablet computer, or smartphone. In some examples, computer system 800 may be a cloud computing node. Computer system 800 may be described in the general context of computer system executable instructions, such as program modules, being executed by a computer system. Generally, program modules may include routines, programs, objects, components, logic, data structures, and so on that perform particular tasks or implement particular abstract data types. Computer system 800 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 4, the computer system 800 has one or more central processing units (CPU(s)) 801a, 801b, 801c, etc. (collectively or generically referred to as processor(s) 801). The processors 801 can be a single-core processor, multi-core processor, computing cluster, or any number of other configurations. The processors 801, also referred to as processing circuits, are coupled via a system bus 802 to a system memory 803 and various other components. The system memory 803 can include one or more memory devices, such as a read-only memory (ROM) 804 and a random access memory (RAM) 805. The ROM 804 is coupled to the system bus 802 and may include a basic input/output system (BIOS), which controls certain basic functions of the computer system 800. The RAM is read-write memory coupled to the system bus 802 for use by the processors 801. The system memory 803 provides temporary memory space for operations of said instructions during operation. The system memory 803 can include random access memory (RAM), read-only memory, flash memory, or any other suitable memory systems.

The computer system 800 comprises an input/output (I/O) adapter 806 and a communications adapter 807 coupled to the system bus 802. The I/O adapter 806 may be a small computer system interface (SCSI) adapter that communicates with a hard disk 808 and/or any other similar component. The I/O adapter 806 and the hard disk 808 are collectively referred to herein as a mass storage 810.

Software 811 for execution on the computer system 800 may be stored in the mass storage 810. The mass storage 810 is an example of a tangible storage medium readable by the processors 801, where the software 811 is stored as instructions for execution by the processors 801 to cause the computer system 800 to operate, such as is described hereinbelow with respect to the various Figures. Examples of computer program product and the execution of such instruction is discussed herein in more detail. The communications adapter 807 interconnects the system bus 802 with a network 812, which may be an outside network, enabling the computer system 800 to communicate with other such systems. In one aspect, a portion of the system memory 803 and the mass storage 810 collectively store an operating system, which may be any appropriate operating system to coordinate the functions of the various components shown in FIG. 4.

Additional input/output devices are shown as connected to the system bus 802 via a display adapter 815 and an interface adapter 816 and. In one aspect, the adapters 806, 807, 815, and 816 may be connected to one or more I/O buses that are connected to the system bus 802 via an intermediate bus bridge (not shown). A display 819 (e.g., a screen or a display monitor) is connected to the system bus 802 by a display adapter 815, which may include a graphics controller to improve the performance of graphics-intensive applications and a video controller. A keyboard, a mouse, a touchscreen, one or more buttons, a speaker, etc., can be interconnected to the system bus 802 via the interface adapter 816, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 4, the computer system 800 includes processing capability in the form of the processors 801, and storage capability including the system memory 803 and the mass storage 810, input means such as the buttons, touchscreen, and output capability including the speaker 823 and the display 819.

In some aspects, the communications adapter 807 can transmit data using any suitable interface or protocol, such as the internet small computer system interface, among others. The network 812 may be a cellular network, a radio network, a wide area network (WAN), a local area network (LAN), or the Internet, among others. An external computing device may connect to the computer system 800 through the network 812. In some examples, an external computing device may be an external web server or a cloud computing node.

It is to be understood that the block diagram of FIG. 4 is not intended to indicate that the computer system 800 is to include all of the components shown in FIG. 4. Rather, the computer system 800 can include any appropriate fewer or additional components not illustrated in FIG. 4 (e.g., additional memory components, embedded controllers, modules, additional network interfaces, etc.). Further, the aspects described herein with respect to computer system 800 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects.

The reports/views/annotations and other information described herein is added to an electronic medical record (EMR) in one or more cases. In some aspects, the information about specific surgical procedures can be stored in the patient record associated with the patient that was operated upon during the surgical procedure. Alternatively, or in addition, the information is stored in a separate database for later retrieval. The retrieval can be associated with the patient's unique identification, such as EMR-identification, social security number, or any other unique identifier. The stored data can be used to generate patient-specific reports. In some aspects, information can also be retrieved from the EMR to enhance one or more operations described herein. In one or more aspects, an operational note may be generated, which includes one or more outputs from the machine learning models. The operational note may be stored as part of the EMR.

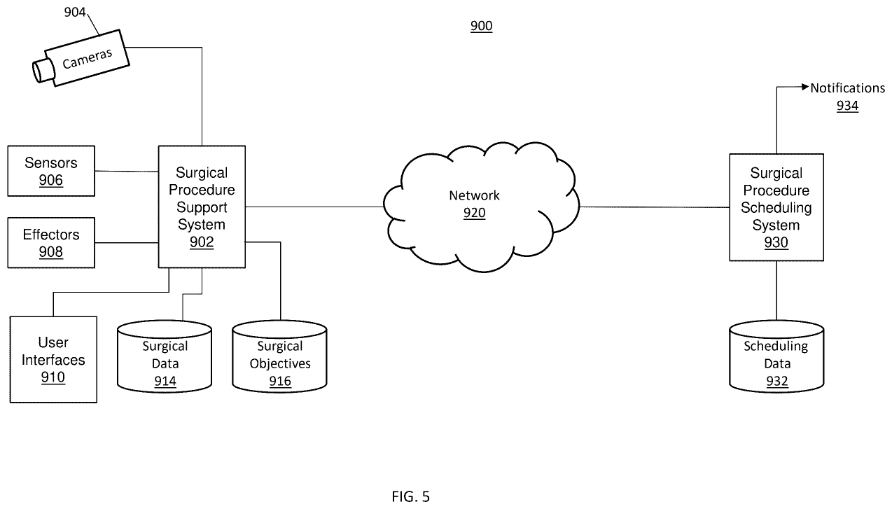

FIG. 5 depicts a surgical procedure system 900 in accordance with one or more aspects. The example of FIG. 5 depicts a surgical procedure support system 902 configured to communicate with a surgical procedure scheduling system 930 through a network 920. The surgical procedure support system 902 can include or may be coupled to the system 100. The surgical procedure support system 902 can acquire image data, such as images, using one or more cameras 904. The surgical procedure support system 902 can also interface with a plurality of sensors 906 and effectors 908. The sensors 906 may be associated with surgical support equipment and/or patient monitoring. The effectors 908 can be robotic components or other equipment controllable through the surgical procedure support system 902. The surgical procedure support system 902 can also interact with one or more user interfaces 910, such as various input and/or output devices. The surgical procedure support system 902 can store, access, and/or update surgical data 914 associated with a training dataset and/or live data as a surgical procedure is being performed. The surgical procedure support system 902 can store, access, and/or update surgical objectives 916 to assist in training and guidance for one or more surgical procedures.

The surgical procedure scheduling system 930 can access and/or modify scheduling data 932 used to track planned surgical procedures. The scheduling data 932 can be used to schedule physical resources and/or human resources to perform planned surgical procedures. Based on the surgical maneuver as predicted by the one or more machine learning models and a current operational time, the surgical procedure support system 902 can estimate an expected time for the end of the surgical procedure. This can be based on previously observed similarly complex cases with records in the surgical data 914. A change in a predicted end of the surgical procedure can be used to inform the surgical procedure scheduling system 930 to prepare the next patient, which may be identified in a record of the scheduling data 932. The surgical procedure support system 902 can send an alert to the surgical procedure scheduling system 930 that triggers a scheduling update associated with a later surgical procedure. The change in schedule can be captured in the scheduling data 932. Predicting an end time of the surgical procedure can increase efficiency in operating rooms that run parallel sessions, as resources can be distributed between the operating rooms. Requests to be in an operating room can be transmitted as one or more notifications 934 based on the scheduling data 932 and the predicted surgical maneuver.

As surgical maneuvers and steps are completed, progress can be tracked in the surgical data 914, and status can be displayed through the user interfaces 910. Status information may also be reported to other systems through the notifications 934 as surgical maneuvers are completed or if any issues are observed, such as complications.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A computer-implemented user interface comprising: a plurality of user interface elements, each user interface element respectively corresponding to a structure from a list of structures anticipated in a surgical video, each user interface element having a visual attribute, wherein the visual attribute of a first user interface element is set to a first state in response to a first structure being detected in the surgical video in a field of view, and the visual attribute of the first user interface element is set to a second state in response to the first structure not being detected in the field of view.

2. The computer-implemented user interface of claim 1, wherein the visual attribute of the first user interface element is based on the first structure being marked with an overlay using said visual attribute.

3. The computer-implemented user interface of claim 1, wherein the first structure is detected in the field of view using machine learning.

4. The computer-implemented user interface of claim 1, wherein the plurality of user interface elements is grouped in a single menu user interface.

5. The computer-implemented user interface of claim 4, wherein the menu user interface is a toolbar displayed as a graphical overlay on the surgical video.

6. The computer-implemented user interface of claim 1, wherein the surgical video is a live video stream.

7. The computer-implemented user interface of claim 5, wherein a location of rendering the toolbar is fixed or user-configurable.

8. The computer-implemented user interface of claim 1, wherein the list of structures comprises a predetermined list of structures based on a type of surgery in the surgical video.

9. The computer-implemented user interface of claim 1, wherein the list of structures comprises a dynamic list of structures.

10. The computer-implemented user interface of claim 1, wherein the visual attribute is one of a color, a pattern, a shape, an image, and an animation.

11. The computer-implemented user interface of claim 1, wherein the user interface element comprises a label.

12. A computer-implemented method comprising: identifying, by one or more processors, a structure in a video of a surgical procedure using machine learning; in response to at least a portion of the structure being visible in a field of view, representing, by the one or more processors, a user interface element corresponding to the structure using a first visual attribute; and in response to the structure not being visible in the field of view, representing, by the one or more processors, the user interface element corresponding to the structure using a second visual attribute.

13. The computer-implemented method of claim 12, wherein the structure is one of an anatomical structure and a surgical instrument.

14. The computer-implemented method of claim 13, wherein the anatomical structure is one of an organ, artery, duct, surgical artifact, and anatomical landmark.

15. The computer-implemented method of claim 13, wherein the surgical instrument is one of clamps, staplers, knives, scalpels, sealers, dividers, dissectors, tissue fusion instruments, monopolars, Marylands, and fenestrated.

16. The computer-implemented method of claim 12, wherein the structure is one from a predetermined list of structures.

17. The computer-implemented method of claim 12, wherein the user interface element comprises a geometric shape that is displayed at a predetermined position during a display of the video of the surgical procedure.

18. The computer-implemented method of claim 12, wherein the first visual attribute is used to represent the structure in response to the structure being in the field of view by displaying a graphical overlay with the same first visual attribute to highlight the structure.

19. The computer-implemented method of claim 12, wherein the video is a live video stream of the surgical procedure.

20. A computer program product comprising a memory device having computer executable instructions stored thereon, which when executed by one or more processors cause the one or more processors to perform a method for generating a user interface to depict presence of structures in a field of view, the method comprising: identifying, using a neural network model, a structure in a video of a surgical procedure, the neural network model is trained using surgical training data; generating a visualization that comprises a graphical overlay at a location of the structure in the video of the surgical procedure, the graphical overlay uses a first visual attribute; identifying a symbol corresponding to the structure from a list of displayed symbols; and updating the symbol by displaying the symbol using the first visual attribute.
